(19) United States
(12) Patent Application Publication
(10) Pub. No.: US 2004/0034062 A1
(43) Pub. Date: Feb. 19, 2004

(54) Novel aqueous anti-inflammatory pharmaceutical formulation

(76) Inventors: Duncan Robert Armour, Stevenage (GB); David Brown, Hertforshire (GB); Miles Stuart Congreve, Cambridge (GB); Paul Martin Gore, Hertfordshire (GB); Darren Victor Green, Hertfordshire (GB); Stuart Holman, Hertfordshire (GB); Andrew McMurtrie Mason, Hertfordshire (GB); Karen Morris, Hertfordshire (GB); Nigel Grahame Ramsden, Hertfordshire (GB); Marian Thomas, Hertfordshire (GB); Peter Ward, Hertfordshire (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/311,297

(22) PCT Filed: Jun. 15, 2001
(86) PCT No.: PCT/GB01/02644

(30) Foreign Application Priority Data
Jun. 16, 2000 (GB) 0014892.4

(51) Int. Cl. A61K 31/445

(52) U.S. Cl. 514/330; 514/57; 514/317

(57) ABSTRACT

The present invention relates to a pharmaceutical formulation which comprises: an aqueous solution of (2S)-3-[4-({[4-(aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid or a salt or solvate thereof. Methods and uses of the formulation in the treatment of allergic rhinitis are also described, as are containers containing said formulation.

The present invention relates to aqueous formulations for use in the treatment of respiratory disorders, in particular to formulations suitable for nasal administration.

(2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid has recently been described in International Patent Application PCT/EP99/10000 (the contents of which are herein incorporated by reference) as a novel antagonist of both α₄β₁ and α₄β₇ integrins which, as a consequence, results in effective anti-inflammatory properties. However, there is a need for a formulation suitable for treatment of inflammatory conditions of the upper respiratory tract, in particular, rhinitis which is an abnormal bodily condition that involves inflammation of the mucous membranes of the nose.

Many millions of individuals suffer from seasonal and perennial allergic rhinitis worldwide. Symptoms of seasonal and perennial allergic rhinitis include nasal itch, congestion, runny nose, sneezing and watery eyes. Seasonal allergic rhinitis is commonly known as ‘hay fever’. It is caused by allergens which are present in the air at specific times of the year, for example tree pollen during Spring and Summer. Perennial allergic rhinitis is caused by allergens which are present in the environment during the entire year, for example dust mites, mold, mildew and pet dander.

To formulate an effective pharmaceutical nasal composition, the medicament must be delivered readily to all portions of the nasal cavities (the target tissues) where it performs its pharmacological function. Additionally, the medicament should remain in contact with the target tissues for relatively long periods of time. The longer the medicament remains in contact with the target tissues, the medicament must be capable of resisting those forces in the nasal passages that function to remove particles from the nose. Such forces, referred to as ‘mucocillary clearance’, are recognised as being extremely effective in removing particles from the nose in a rapid manner, for example, within 10-30 minutes from the time the particles enter the nose.

Other desired characteristics of a nasal composition are that it must not contain ingredients which cause the user discomfort, that it has satisfactory stability and shelf-life properties, and that it does not include constituents that are considered to be detrimental to the environment, for example ozone depletors.

Thus, according to the present invention we provide a pharmaceutical formulation which comprises:

an aqueous solution of (2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid or a salt or solvate thereof.

Preferably, the formulation will contain one or more viscosity enhancing agents.

Preferably, the formulation will contain one or more preservatives.

Preferably, the formulation will contain one or more isotonicity adjusting agents.

According to one particular aspect of the present invention we provide a pharmaceutical formulation which comprises:

(i) an aqueous solution of (2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid or a salt or solvate thereof;

(ii) one or more viscosity enhancing agents;

(iii) one or more preservatives;

(iv) one or more isotonicity adjusting agents.

The formulations of the present invention may be stabilised by appropriate selection of pH using hydrochloric acid. Typically, the pH will be adjusted to between 4.5 and 7.5, preferably between 5.0 and 6.0, especially 5.5.

Examples of pharmaceutically acceptable materials which can be used to adjust the pH of the formulation include hydrochloric acid and sodium hydroxide. Preferably, the pH of the formulation will be adjusted using hydrochloric acid.

The aqueous component is preferably a high grade quality of water, most preferably purified water.

Examples of suitable salts include physiologically acceptable salts such as alkali metal salts, for example calcium, sodium and potassium salts and salts with (trishydroxymethyl)aminomethane.

Of particular interest as medicament is (2S)-3-[4-({[4-(Aminocarbonyl)-1-piperidinyl]carbonyl}oxy)phenyl]-2-[((2S)-4-methyl-2-{[2-(2-methylphenoxy) acetyl]amino}pentanoyl)amino] propanoic acid potassium salt. Also of interest is the free acid.

A pharmaceutically acceptable amount of (2S)-3-[4-({[4-(Aminocarbonyl)-1-piperidinyl]carbonyl}oxy)phenyl]-2-[((2S)-4-methyl-{[2-(2-methylphenoxy) acetyl]amino}pentanoyl)amino] propanoic acid or a salt or solvate thereof is present within the formulation, in an amount which is preferably between 0.1% and 20% (w/w), preferably between 0.5% and 5% (w/w), based on the total weight of the formulation. Typically, 100 μl of the solution formulation will contain 1 mg of (2S)-3-[4-({[4-(Aminocarbonyl)-1-piperidinyl]carbonyl}oxy) phenyl]-2-[((2S)-4-methyl-2-{[2-(2-methylphenoxy)acetyl]amino}pentanoyl)amino] propanoic acid or a salt thereof.

Examples of viscosity enhancing agents include carboxymethylcellulose, veegum, tragacanth, bentonite, methylcellulose, poloxamers (eg. poloxamer 407) and polyethylene glycols. Preferably, the viscosity enhancing agent will be microcrystalline cellulose and carboxy methylcellulose sodium, most preferably used as the branded product Avicel RC591 (which typically contains 87-91% microcrystalline cellulose and 9-13% carboxy methylcellulose sodium). Particulate microcrystalline cellulose will preferably have a particle size in the range 1 to 100 μm. We believe that Avicel RC591 acts as a viscosity enhancing agent by imparting thixotropic properties to the formulation.

Preferably, the viscosity enhancing agent will possess thixotropic properties which will ensure that the formulation assumes a gel like appearance at rest, characterised by a high viscosity value. Once the composition is subjected to shear forces, such as those caused by agitation prior to spraying, the viscosity of the formulation will preferably decrease to such a level to enable it to flow readily through the spray device and exit as a fine mist spray. This mist will then be capable of infiltrating the mucosal surfaces of the anterior regions of the nose (frontal nasal cavities), the frontal sinus, the maxillary sinuses and the turbinates which overlie the conchas of the nasal cavities. Once deposited, the viscosity of the formulation will preferably increase to a sufficient level to assume its gel-like form and resist being cleared from the nasal passages by the inherent mucocillary forces that are present in the nasal cavities.

When the formulation of the present invention comprises a viscosity enhancing agent, it will be desirably added in a suitable amount to achieve this function, preferably the viscosity enhancing agent will be present within the formulation in an amount of between 0.1 and 5% (w/w), especially 1.5% (w/w), based on the total weight of the formulation.

For stability purposes, the formulation of the present invention should be protected from microbial contamination and growth. Examples of pharmaceutically acceptable anti-microbial agents that can be used in the formulation include quaternary ammonium compounds (eg. benzalkonium chloride, benzethonium chloride, cetrimide and cetylpyridinium chloride), mercurial agents (eg. phenylmercuric nitrate, phenylmercuric acetate and thimerosal), alcoholic agents (eg. chlorobutanol, phenylethyl alcohol and benzyl alcohol), antibacterial esters (eg. esters, of para-hydroxybenzoic acid) and other anti-microbial agents such as chlorhexidine, chlorocresol and polymyxin.

Preferably, the preservative will comprise phenylethyl alcohol, which will preferably be present within the formulation in an amount of between 0.05 and 5% (v/w), especially 0.25% (v/w), based on the total weight of the formulation.

Preferably, the preservative will comprise benzalkonium chloride, which will preferably be present within the formulation in an amount of between 0.001 and 1% (w/w), especially 0.02% (w/w), based on the total weight of the formulation.

More preferably, the preservative comprises phenylethyl alcohol and benzalkonium chloride.

The presence of an isotonicity adjusting agent is to achieve isotonicity with body fluids eg fluids of the nasal cavity, resulting in reduced levels of irritancy associated with many nasal formulations. Examples of suitable isotonicity adjusting agents are sodium chloride, dextrose and calcium chloride. Preferably, the isotonicity adjusting agent will be dextrose, most preferably used as dextrose anhydrous.

When the formulation of the present invention comprises an isotonicity adjusting agent it will be desirably added in a sufficient quantity to achieve this function, preferably the isotonicity adjusting agent will be present within the formulation in an amount of between 0.1 and 10% (w/w), especially 5.0% w/w, based on the total weight of the formulation.

Optionally a further active ingredient may be incorporated into the formulation especially one suitable for nasal delivery such as a corticosteroid (eg fluticasone propionate) or an anti-histamine (eg loratadine).

Typically, the formulation of the present invention will be packaged into a suitable container, eg. a multi-dose container with a nasal applicator, wherein the dose is capable of being metered by volume.

Preferable means for applying the formulation of the present invention to the nasal passages is by use of a pre-compression pump. Most preferably, the pre-compression pump will be a VP7 model manufactured by Valois SA. Such a pump is beneficial as it will ensure that the formulation is not released until a sufficient force has been applied, otherwise smaller doses may be applied. Another advantage of the pre-compression pump is that atomisation of the spray is ensured as it will not release the formulation until the threshold pressure for effectively atomising the spray has been achieved. Typically, the VP7 model is capable of holding 16.5 ml of a formulation. Each spray will typically deliver 100 μl of such a formulation, therefore, the VP7 model is capable of providing at least about 120 metered doses.

A suitable dosing regime for the formulation of the present invention when administered to the nose would be for the patient to inhale deeply subsequent to the nasal cavity being cleared. During inhalation the formulation would be applied to one nostril while the other is manually compressed. This procedure would then be repeated for the other nostril.

Preferably, the formulation of the present invention will contain between 0.1 and 20 mg of (2S)-3-[4-({[4-(Aminocarbonyl)-1-piperidinyl]carbonyl} oxy) phenyl]-2-[((2S)-4-methyl-2-[2-(2-methylphenoxy)acetyl]amino}pentanoyl)amino] propanoic acid (or a salt or solvate thereof) per dose, most preferably between 0.5 and 5 mg per dose.

Typically, one or two inhalations per nostril would be administered by the above procedure up to three times each day.

It will be appreciated that the above dosing regime should be adjusted according to the patient's age, body weight and/or symptom severity.

Examples of disease states in which the formulation of the present invention has potentially beneficial anti-inflammatory effects include allergies associated with the nasal cavity, more particularly allergic rhinitis.

Thus, according to a further aspect of the invention we provide a pharmaceutical formulation of the present invention for use in the treatment or prophylaxis of allergic rhinitis.

We also provide a use of a pharmaceutical formulation of the present invention in the manufacture of a medicament for the treatment or prophylaxis of allergic rhinitis.

We also provide a method of treatment of allergic rhinitis which comprises administering to a patient a pharmaceutically acceptable amount of the formulation of the present invention.

More specifically, the formulation of the present invention may be illustrated by reference to the following examples:

### EXAMPLE A

(2S)-3-[4-({[4-(Aminocarbonyl)-1-piperidinyl]carbonyl} oxy) phenyl]-2-[((2S)-4-methyl-2-{[2-(2-methylphenoxy)acetyl]amino}pentanoyl)amino] propanoic acid potassium salt

To Wang resin (50 g) was added a solution of (2S)-3-[4-(allyloxy)phenyl]-2-[(tert-butoxycarbonyl)amino] propanoic acid (115.8 g) and 1-hydroxybenzotriazole (48.6 g) in DMF (475 ml). After 15 minutes 1,3-diisopropylcarbodiimide (56.5 ml) was added and the mixture was stirred for 24 h at 45° C. The resin was filtered and washed with DMF (3×360 ml), methanol (3×360 ml) and dichloromethane (3×700 ml). To a slurry of the resin in dichloromethane (644 ml) was added pyridine (14.7 ml). Acetic anhydride (26.9 ml) was added and the mixture was stirred for 12 h at 20° C. The resin was filtered and washed with dichloromethane (3×550 ml), methanol (3×370 ml) and dichloromethane (3×550 ml).

A slurry of 20 g of the resin in dichloromethane (100 ml) was cooled to 2-5° C. and treated with a solution of phenol (20 g) in dichloromethane (80 ml). Chlorotrimethylsilane (20 ml) was added dropwise and the mixture was stirred for 6 h at 2-5° C. The resin was filtered and washed with dichloromethane (3×200 ml), methanol (3×200 ml), 10% water in DMF (2×200 ml), 10% diisopropylethylamine in DMF (3×200 ml), DMF (200 ml), methanol (3×200 ml) and dichloromethane (3×200 ml).

A slurry of the resin in DMF (55 ml) was treated with a solution of Fmoc-leucine (32.7 g) and 1-hydroxybenzotriazole (12.5 g) in DMF (85 ml). After 5 minutes 1,3-diisopropylcarbodiimide (19.3 ml) was added and the mixture was stirred for 15 h at 20° C. The resin was filtered and washed with DMF (3×150 ml), methanol (3×150 ml) and dichloromethane (3×150 ml).

The resin was treated with 20% piperidine in DMF (180 ml) and stirred for 1 h at 20° C. The resin was filtered and washed with DMF (3×150 ml), dichloromethane (3×150 ml), DMF (3×150 ml) and dichloromethane (3×150 ml). To a slurry of this in DMF (50 ml) was added a solution of (2-methylphenoxy)acetic acid (17.9 g) and 1-hydroxybenzotriazole (14.6 g) in DMF (100 ml). After 5 minutes 1,3-diisopropylcarbodiimide (16.9 ml) was added and the mixture was stirred for 65 h at 20° C. The resin was filtered and washed with DMF (2×150 ml), methanol (3×150 ml) and dichloromethane (3×150 ml).

A slurry of the resin in dichloromethane (60 ml) was treated with a solution of tetrakis(triphenylphosphine)palladium(0) (5.21 g) in dichloromethane (140 ml) followed by morpholine (13 ml). The mixture was stirred for 2 h at 20° C. then the resin was filtered and washed with dichloromethane (7×200 ml).

A slurry of the resin in dichloromethane (160 ml) was treated with diisopropylethylamine (12.4 ml) followed by 4-nitrophenyl chloroformate (24.8 g) in 3 portions at 5 minute intervals. The mixture was stirred for 1 h at 20° C. The resin was filtered and washed with dichloromethane (3×200 ml). The resin was treated with a solution of isonipecotamide (15.8 g) in DMF (180 ml) and the mixture was stirred for 1.5 h at 20° C. The resin was filtered and washed with DMF (4×200 ml) and dichloromethane (2×200 ml).

The resin was treated with 50% TFA in dichloromethane (200 ml). After stirring for 1 h at 20° C. the resin was filtered and washed with dichloromethane (5×200 ml). The combined filtrate and washings were evaporated in vacuo. The residue was azeotroped with toluene (2×100 ml) then triturated with ether (50 ml) and the resulting white solid filtered. To this was added acetonitrile (150 ml) and the mixture was heated to reflux. The resulting suspension was allowed to cool to 20° C. and stirred for 18 h. The mixture was filtered to give (2S)-3-[4-({[4-(Aminocarbonyl)-1-piperidinyl]carbonyl} oxy) phenyl]-2-[((2S)-4-methyl-2-{[2-(2-methylphenoxy)acetyl]amino}pentanoyl) amino] propanoic acid as a white solid (4.9 g).

A suspension of (2S)-3-[4-({[4-(Aminocarbonyl)-1-piperidinyl]carbonyl} oxy) phenyl]-2-[((2S)-4-methyl-2-{[2-(2-methylphenoxy)acetyl]amino}pentanoyl) amino] propanoic acid (10 g) in methanol (150 ml) was warmed to reflux to obtain a clear solution. To this was added a solution of potassium carbonate (1.16 g) in water (7.5 ml). After heating under reflux for two minutes the solvents were evaporated in vacuo to give a crisp foam. To this was added acetonitrile (100 ml) and the mixture was warmed to reflux, during which time the foam collapsed and started to crystallise. After ten minutes the mixture was allowed to cool to 20° C. then filtered under reduced pressure, washed-with acetonitrile (25 ml) and ether (50 ml) to give the title compound as a white solid (10.65 g, 100%).

### Example 1

| | |
|---|---|
| (2S)-3-[4-({[4-(Aminocarbonyl)-1-piperidinyl]carbonyl}- | 1% (w/w) |
| oxy)phenyl]-2-[((2S)-4-methyl-2-{[2-(2-methylphenoxy)- | |
| acetyl]amino}pentanoyl)amino]propanoic acid | |
| (prepared according to Example A) | |
| Phenylethyl alcohol | 0.25% (v/w) |
| Microcrystalline cellulose | 1.5% (w/w) |
| and carboxymethylcellulose sodium (Avicel RC591) | |
| Benzalkonium chloride | 0.02% (w/w) |
| Hydrochloric acid | to pH 5.5 |
| Purified water | to 100%. |

In a 100 μl metered volume dispensed by a Valois VP7 pre-compression pump, approximately 1 mg of (2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid potassium salt will be delivered.

### Example 2

| | |
|---|---|
| (2S)-3-[4-({[4-(Aminocarbonyl)-1-piperidinyl]carbonyl}- | 1% (w/w) |
| oxy)phenyl]-2-[((2S)-4-methyl-2-{[2-(2-methylphenoxy)- | |
| acetyl]amino}pentanoyl)amino]propanoic acid | |
| (prepared according to Example A) | |
| Phenylethyl alcohol | 0.25% (v/w) |
| Microcrystalline cellulose | 1.5% (w/w) |
| and carboxymethylcellulose sodium (Avicel RC591) | |
| Benzalkonium chloride | 0.02% (w/w) |
| Dextrose anhydrous | 5.0% (w/w) |
| Hydrochloric acid | to pH 5.5 |
| Purified water | to 100%. |

In a 100 μl metered volume dispensed by a Valois VP7 pre-compression pump, approximately 1 mg of (2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid potassium salt will be delivered.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

The contents of the above mentioned patent applications are herein incorporated by reference.

1. A pharmaceutical formulation which comprises:
an aqueous solution of (2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid or a salt or solvate thereof.

2. A pharmaceutical formulation according to claim 1 which comprises:
one or more viscosity enhancing agents.

3. A pharmaceutical formulation according to claim 2 wherein the viscosity enhancing agent is microcrystalline cellulose and carboxy methylcellulose sodium.

4. A pharmaceutical formulation according to claim 2 or claim 3 wherein the viscosity enhancing agent is present in an amount of between 0.1 and 5% (w/w), based on the total weight of the formulation.

5. A pharmaceutical formulation according to any one of claims 1 to 4 which comprises:
one or more preservatives.

6. A pharmaceutical formulation according to claim 5 wherein the preservative comprises phenylethyl alcohol.

7. A pharmaceutical formulation according to claim 6 wherein the phenylethyl alcohol is present within the formulation in an amount of between 0.05 and 5% (v/w), based on the total weight of the formulation.

8. A pharmaceutical formulation according to claim 5 wherein the preservative comprises benzalkonium chloride.

9. A pharmaceutical formulation according to claim 8 wherein the benzalkonium chloride is present within the formulation in an amount of between 0.001 and 1% (w/w), based on the total weight of the formulation.

10. A pharmaceutical formulation according to any one of claims 5 to 9 wherein the preservative comprises phenylethyl alcohol and benzalkonium chloride.

11. A pharmaceutical formulation according to any one of claims 1 to 10 which comprises:
one or more isotonicity adjusting agents.

12. A pharmaceutical formulation according to claim 11 wherein the isotonicity adjusting agent is dextrose.

13. A pharmaceutical formulation according to claim 12 wherein dextrose is present within the formulation in an amount of between 0.1 and 10% (w/w), based on the total weight of the formulation.

14. A pharmaceutical formulation according to any one of claims 1 to 13 characterised in that it is isotonic with fluids of the nasal cavity.

15. A pharmaceutical formulation according to any one of claims 1 to 14 which is buffered to a pH of between 5 and 7.

16. A pharmaceutical formulation according to claim 15 which is buffered using hydrochloric acid.

17. A pharmaceutical formulation according to any one of claims 1 to 16 wherein (2S)-3-[4-({[4-(Aminocarbonyl)-1-piperidinyl]carbonyl}oxy) phenyl]-2-[((2S)-4-methyl-2-{[2-(2-methylphenoxy)acetyl]amino}pentanoyl)amino] propanoic acid or a salt or solvate thereof is present within the formulation in an amount between 0.1% and 20% (w/w), based on the total weight of the formulation.

18. A pharmaceutical formulation according to claim 17 wherein (2S)-3-[4-({[4-(Aminocarbonyl)-1-piperidinyl]carbonyl}oxy) phenyl]-2-[((2S)-4-methyl-2-{[2-(2-methylphenoxy)acetyl]amino}pentanoyl)amino] propanoic acid or a salt or solvate thereof is present within the formulation in an amount of between 0.5% and 5% (w/w), based on the total weight of the formulation.

19. A pharmaceutical formulation according to any one of claims 1 to 18 wherein (2S)-3-[4-({[4-(Aminocarbonyl)-1-piperidinyl]carbonyl}oxy) phenyl]-2-[((2S)-4-methyl-2-{[2-(2-methylphenoxy)acetyl]amino} pentanoyl)amino] propanoic acid is present as the potassium salt.

20. A container comprising a pharmaceutical formulation according to any one of claims 1 to 19 suitable for delivering it in the form of a nasal spray.

21. A pharmaceutical formulation according to any one of claims 1 to 19 for use in the treatment or prophylaxis of allergic rhinitis.

22. Use of a pharmaceutical formulation according to any one of claims 1 to 19 in the manufacture of a medicament for the treatment or prophylaxis of allergic rhinitis.

23. A method of treatment of allergic rhinitis which comprises administering to a patient a pharmaceutically acceptable amount of a formulation according to claims 1 to 19.
